(19) United States
(12) Patent Application Publication
(10) Pub. No.: US 2008/0283449 A1
(43) Pub. Date: Nov. 20, 2008

(54) Method of Analyzing and Sorting Eggs

(75) Inventor: Jakob Find Madsen, Glostrup (DK)

Correspondence Address: DYKEMA GOSSETT PLLC, FRANKLIN SQUARE, THIRD FLOOR WEST, 1300 I STREET, NW, WASHINGTON, DC, 20005, US

(73) Assignee: IMAGE HOUSE A/S, Copenhagen K (DK)

(21) Appl. No.: 11/666,044

(22) PCT Filed: Oct. 12, 2005
(86) PCT No.: PCT/DK05/00652

§ 371 Date: Jun. 18, 2007

(30) Foreign Application Priority Data
Oct. 22, 2004 (DK) PA 2004 01622

(51) Int. Cl. G01N 33/08

(52) U.S. Cl. 209/511

(57) ABSTRACT

In a method of analyzing and sorting eggs which are advanced and rolled on a conveyor belt, a plurality of images en of each egg are recorded by a plurality of cameras, and it is analyzed for each egg whether there are any areas in the image which may be an indication of whether the egg is cracked, contains impurities or the like. The analysis takes place in that each image is subjected to a line scan, and if the line includes an edge defined by two adjacent pixels where the intensity value between the two adjacent pixels is numerically greater than a certain value, e.g. 15, a discriminant analysis is provided around the edge in a subarea 3 of the image of 25×25 pixels. The analysis establishes a class which defines characteristic features around the edge. When all the edges of the egg have been analyzed, it is determined by a calculation of the classes, including whether the classes define clusters, whether the egg has a crack, an impurity or the like, which means that it is removed by means of a robot. The invention provides a high degree of precision in the sorting-out of eggs which have tracks, impurities or the like, as it is not the total number of pixels with light intensities above a certain value on the entire egg which is used as a criterion, but an evaluation of the position of the edges and characteristic features on the surface of the eggs which is used for the analyses.

The invention relates to a method of analyzing and sorting eggs which are transported on a conveyor belt wherein the eggs are advanced and rolled about their axes on the conveyor belt, and wherein a plurality of cameras record images of the eggs which are analyzed at pixel level for cracks, impurities and the like, and the eggs having cracks or impurities of a certain size are sorted out.

Such a system is known from U.S. Pat. No. 6,433,293. In this known system, the analysis is performed in that all the pixels on the surface of the individual egg are analyzed, which means that eggs having many, but very small areas of impurities may undesirably be sorted out.

Accordingly, an object of the invention is to provide a method, wherein the analysis is more accurate such that eggs which do not have “true” impurities are removed.

The object of the invention is achieved by a method of the type defined in the introductory portion of claim 1, which is characterized by
a) recording a plurality of images of each egg,
b) performing a plurality of line scans in each image, where the intensity level of each pixel along the line is measured,
c) analyzing the edges defined by two adjacent pixels on the line where there is a difference in intensity level between the two adjacent pixels which is greater or smaller than a given value, by subjecting a defined area of the image around the edge to a texture analysis in which the defined areas are, divided into classes, said classes being indicative of characteristic features of the egg.

This ensures a very accurate method, as the distribution of pixels with the given features is included in the decision of whether an egg is to be removed from the conveyor belt.

An expedient way of performing the texture analysis is, as stated in claim 2, that it is performed in an area of 25×25 pixels, where the intensity values of the individual pixels are measured, as this has been found to give good results in the analysis of edges.

To refine the method additionally, it is advantageous if, as stated in claim 3, the intensity values are subjected to a discriminant analysis in which the types of the classes are determined, and, as stated in claim 4, that the classes are transformed into a vector having a plurality of parameters which are indicative of the distribution of characteristic features of the eggs.

To ascertain rapidly whether an egg has undesired impurities on its surface, it is expedient if, as stated in claim 5, it is examined whether the cracks, the impurities and the like are disposed close together, and if so a cluster is set up.

In terms of calculation, as stated in claim 6, the method may be performed in that the specific classes and clusters are allocated weights, followed by a calculation of a figure which provides an indication of whether an egg is dirty or destroyed.

To ensure that eggs of different sizes are subjected to the same analysis, it is advantageous if, as stated in claim 7, the number of images recorded covers the entire surface of the individual eggs, the image recording speed of the cameras being synchronized with the speed of rotation and the size of the individual eggs.

Control of the speed of rotation is performed, as stated in claim 8, in that the synchronization of the image recording speed of the camera is determined on the basis of the diameters of the individual eggs.

To ensure that the analysis does not analyze brown and white eggs differently, it is an advantage if, as stated in claim 9, the images are recorded at light having a wavelength of 685-695 nm, preferably 692 nm.

The invention will now be explained more fully with reference to the drawing, in which

FIG. 1 shows a sketch of the system for the analysis of eggs according to the invention,

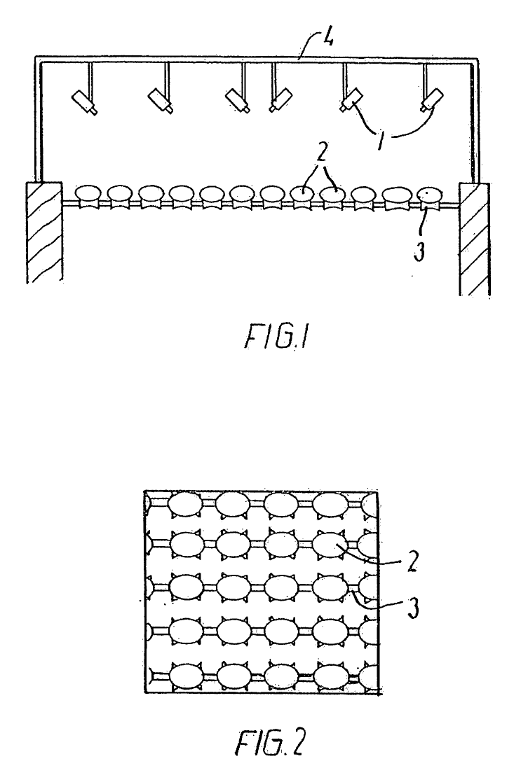

FIG. 2 shows a section of a plurality of eggs on a conveyor device seen from above in FIG. 1,

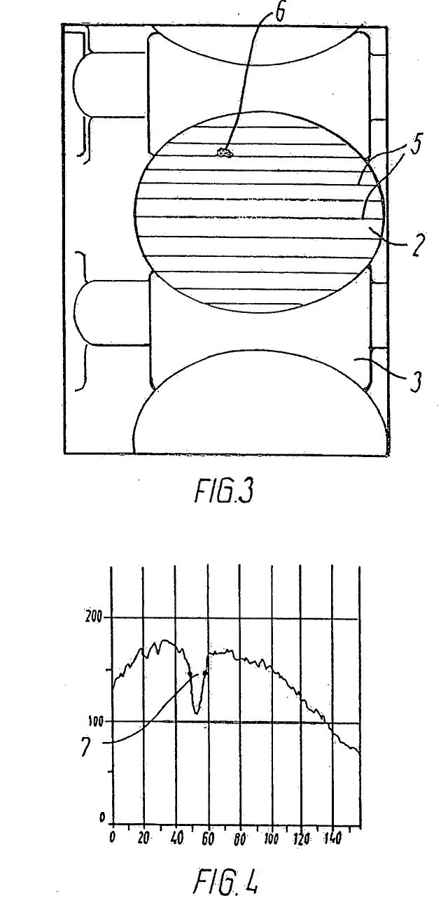

FIG. 3 shows an egg with plotted lines representing lines along which scanning is performed,

FIG. 4 shows an example of a graph produced by the scanning of one of the lines shown in FIG. 3,

FIG. 5 shows an egg with an area which is to be analyzed more closely,

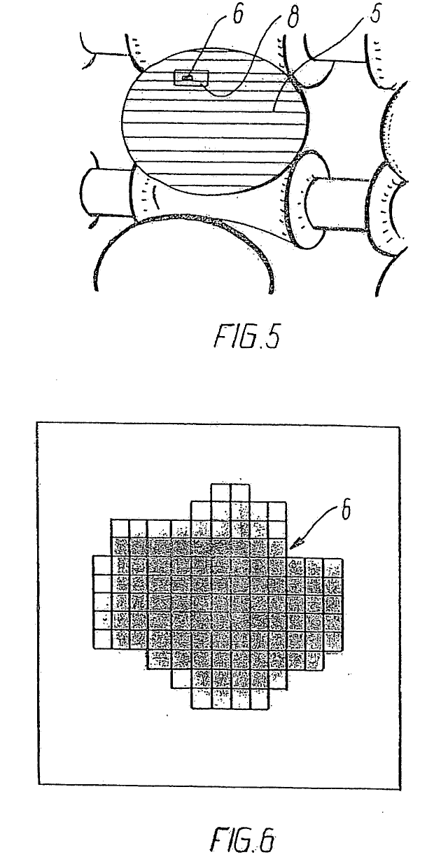

FIG. 6 shows an enlarged view of the area of the egg in FIG. 5,

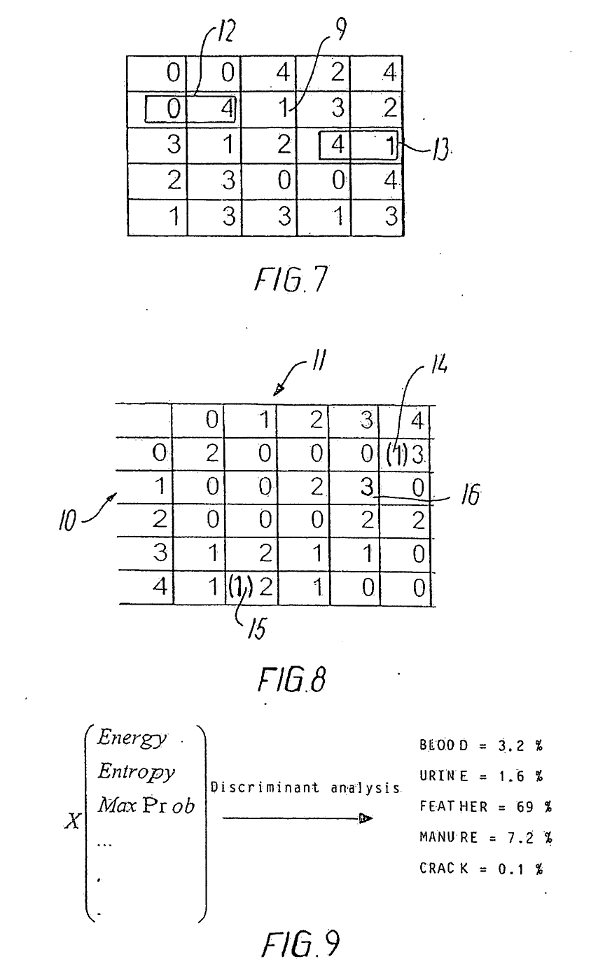

FIG. 7 shows an example of a section of image processing data of the area shown on the egg in FIG. 6,

FIG. 8 shows image processing data in FIG. 7 after a transformation,

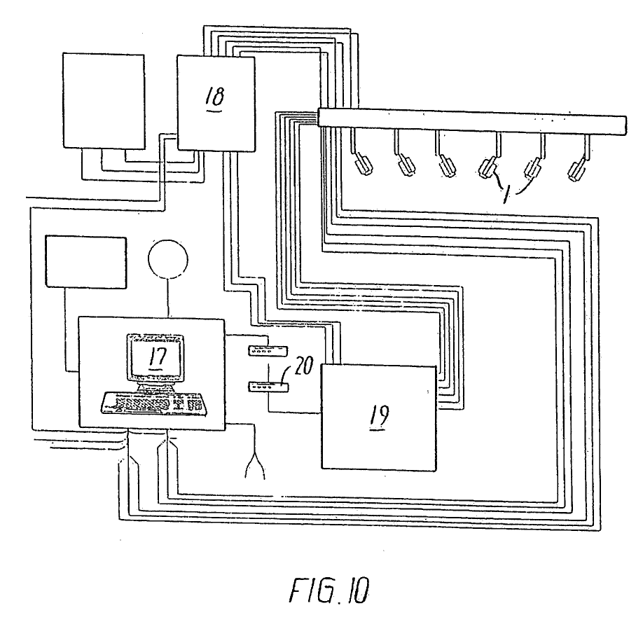

FIG. 9 shows all the data for a spot in vector form, while

FIG. 10 shows a system for performing the invention.

FIG. 1 shows six cameras, of which two are designated 1. The cameras, are suspended from a frame 4 and are intended to record images of eggs 2, which are placed on an underlying conveyor belt.

As will be seen; there is a total of 12 eggs on the underlying conveyor belt, which pass the cameras at a speed of up to 14 cm/s.

In addition to being transported past the cameras, the eggs roll about their own axis, as a transverse roller 3 on the conveyor belt provides the rolling.

The images are recorded such that each camera 1 records a plurality of images, preferably four, of four eggs, so that the entire surface of the egg is recorded. Images of each egg are recorded by two cameras which record in their respective directions.

In general, the images are recorded in light having-a wavelength of 690 nm, so that the analyses are not affected by whether brown or white eggs are analyzed.

Since the eggs may have a varying size, the cameras are set to record images in time intervals which are calculated on the basis of the diameters of the eggs. Also, the positions of the individual eggs are determined so that they may be identified at a later, optional sorting.

The entire set-up is intended to analyze the eggs for impurities, which may be stained spots from dirt, feather residues, cracks and the like, so that they may be removed for another purpose than direct sale to the consumers. The eggs having defects are removed from the conveyor belt by a robot (not shown).

It will now be explained how the analysis according to the invention is performed.

FIG. 3 shows a single egg 2 which has a spot 6. One of the cameras has recorded the image of a portion of the surface of the egg. This image is analyzed by performing a line scan along the lines which are designated 5, and of which there may be fifteen. The scan is performed by measuring the intensity level or grey tone level of each pixel which is present on the lines.

FIG. 4 shows the result of a scan along the line where the spot 6 is present. Exactly where the spot is present, there is a strong reduction and a strong increase in the intensity level, which is shown at 7 in FIG. 4, which means that two adjacent pixels have different intensity levels, shown in the figure by two spots.

As it cannot readily be decided whether the spot means that the egg is to be removed from the conveyor belt, a further analysis is performed, as an area 8, cf. FIGS. 5 and 6, around the spot 6 is subjected to further analysis. The area may have a size which corresponds to 25×25 pixels. The intensity level of each individual pixel inside the area 8 is analyzed, and a result of this is shown in FIG. 7, which shows a table of numerical values 9 representing the intensity level of the measured pixels, it being noted that only an area of 5×5 pixels is shown here. The numeral value 0 indicates a black spot, while the numeral value 4 indicates a white spot.

Thus, analysis of various spots on the eggs will result in the generation of tables which may be used for classifying the spots, since each spot may be described by their table values.

To analyze the spots additionally, a further analysis may be, performed, as explained in connection with FIGS. 7 and 8, for each table found on the basis of one or more spots.

In FIG. 8, the numeral 10 designates a column containing the intensity values 0, 1, 2, 3, 4, while the numeral 11 designates a row containing the intensity values 0, 1, 2, 3, 4.

In FIG. 7, the numerals 12 and 13 designate two adjacent pixels, where 12 has the values 0 and 4, while 13 has the values 4 and 1.

Assuming that nothing is listed in the table in FIG. 8, the following steps are performed:

The adjacent pixels 12 having the values 0 and 4 are introduced into the table in FIG. 8 with a bracketed figure one shown at 14, as 0 is included in the column 10, while 4 is included in the row 11.

The adjacent pixels 13 are introduced into the table in FIG. 8 with a bracketed figure one shown at 15 in FIG. 8.

This operation is repeated for all adjacent pixels, whereby the table in FIG. 8 is generated on the basis of the table in FIG. 7.

The table 8 is interpreted in the following manner:

The FIG. 3 shown at 16 means that there are three pairs of adjacent pixels having the values 1 and 3, which may be seen in FIG. 7.

It is noted that other pairs of figures than the pairs of adjacent pixels 12, 13 may be used for the analysis. It might e.g. be pairs of figures which are disposed diagonally.

The distribution of the values in the table in FIG. 8 provides information on characteristic features of a spot which may be used for a thorough analysis of the individual spots.

The numerical values in the table in FIG. 8 may be set up as a vector X, cf. FIG. 9, with a plurality of parameters, which, for each spot, indicates the probability of the spot being blood, urine, feather, manure, crack or the like.

Then an equation is made with the formula:

Impurity=k1×Σblood+k2Σurine+ . . . k3×ΣY+C

wherein k1, k2, k3 are constants, Y is an additional characteristic value of an impurity, and C is a cluster value which is produced by calculating how close the individual points are to each other.

The figure produced by the equation is used for deciding whether an egg is to be removed from the conveyor belt.

When the analysis is followed as described, it is ensured that only eggs having real defects are sorted out, while eggs having many defects, where the individual defects are of a small size and cannot be seen by the consumers, are not sorted out.

A system for performing the analyses described above may be constructed as shown in FIG. 10, which also shows the cameras 1. The system has a PC 17 coupled by USB gates with amplifiers 20 to a controller 19 which controls the cameras 1, and a light controller 18 which controls some lamps (not shown) to emit flashlight to the eggs which pass the cameras 1.

Such a system may e.g. sort 120,000 eggs, which are distributed on 12 lanes, per hour.

1. A method of analyzing and sorting eggs which are transported on a conveyor belt, wherein the eggs are advanced and rolled about their axes on the conveyor belt, and wherein a plurality of cameras record images of the eggs which are analyzed at pixel level for cracks, impurities and the like, and the eggs having cracks or impurities of a certain size are sorted out, and recording a plurality of images of each egg, and comprising the following steps:
a) performing a plurality of line scans in each image, where the intensity level of each pixel along the line is measured, and
b) analyzing the edges defined by two adjacent pixels on the line where there is difference in intensity level between the two adjacent pixels which is greater or smaller than a given value, by subjecting a defined area of the image around the edge to a texture analysis in which the defined areas are divided into classes, said classes being indicative of characteristic feature of the egg.

2. A method according to claim 1,
comprising performing the texture analysis in an area of 25×25 pixels, where the intensity values of the individual pixels are measured.

3. A method according to claim 2,
comprising subjecting the intensity values to a discriminant analysis in which the types of the classes are determined.

4. A method according to claim 3,
comprising transforming the classes into a vector having a plurality of parameters which are indicative of the distribution of characteristic features of the individual eggs.

5. A method according to claim 1, comprising examining whether the cracks and the impurities are disposed close together, and if so a cluster is set up.

6. A method according to claim 5,
comprising allocating weights to the specific classes and clusters, followed by a calculation of a figure which provides an indication of whether an egg is dirty or destroyed.

7. A method according to claim 1, wherein the number of images recorded covers the entire surface of the individual eggs, the image recording speed of the cameras being synchronized with the speed of rotation and the size of the individual eggs.

8. A method according to claim 3, comprising determining the synchronization of the image recording speed of the camera on the basis of the diameters of the individual eggs.

9. A method according to claim 1, comprising recording the images light in having a wavelength of 685-695 nm.
